(19) United States
(12) Patent Application Publication
(10) Pub. No.: US 2004/0087918 A1
(43) Pub. Date: May 6, 2004

(54) Gaskets suction canister valve

(76) Inventors: H.R. Buster Johnson III, Broken Arrow, OK (US); John Holland, Tyler, TX (US); Brad Sanford, Jacksonville, TX (US); Robert Horick, Tyler, TX (US); Ben Poole, Whitehouse, TX (US); Doug Cundieff, Jacksonville, TX (US)

Correspondence Address: Kimberly Diliberti, Paralegal, Allegiance Corporation, 1430 Waukegan Road, McGaw Park, IL, 60085, US

(21) Appl. No.: 10/286,620

(22) Filed: Nov. 4, 2002

(51) Int. Cl. A61M 1/00

(52) U.S. Cl. 604/319

(57) ABSTRACT

A gasketless valve for use, among other applications, with a vacuum suction canister or bottle that is adapted to collect fluids, such as for purposes of example without limitation, fluids resulting from various medical procedures including surgery as well as wound irrigation applications, to name a few of many possible applications. The gasketless valve can be integrated with a canister lid that incorporates a valve seat that is formed with a smoothly finished sealing ramp or valving surface or face that can surround a vacuum source port adapted to communicate fluid to establish a vacuum within the canister. The gasketless valve cooperates with a valve sealing float of a preferably dissimilar material that is formed with a multiply tapered or variably graduated rim or extent configured to be telescopingly received on the sealing ramp or valving surface to establish a fluid barrier portion to interrupt fluid communication through the vacuum source port as the suction canister becomes filled with collected fluid. The preferred gasketless valve arrangements are preferably adapted for compatibility with the most widely used vacuum pressure sources found in medical and other industrial environments including, for example, vacuum pressure sources ranging from between about 1 and 28 inches of mercury.

### TECHNICAL FIELD

This invention relates to the field of vacuum and suction bottle and canister valves that are most commonly used in medical applications where various fluids, surgical field debris, wound related fluids, and other materials and substances are to be removed and/or aspirated away from a source location to a suction canister or vacuum bottle or container. More specifically, the present invention is directed to a gasketless valve design that is incorporated into such vacuum bottles and suction canisters and which vastly improves the state of the art.

### BACKGROUND OF THE INVENTION

A need to evacuate, aspirate, and remove fluids and various materials and substances from one place to collect and retain such in a container for later analysis and disposal has been prevalent in various industries for many decades. In modern day industrial and medical environments, myriad applications create waste and excess fluids and debris that must be evacuated, aspirated, and removed from a process location or an operative site. Once transferred, such excess fluids and debris must be safely and hygienically collected and retained at a remote location in a suitable container or vessel until any needed analysis thereof can be completed and the collected materials can be disposed of in a manner suited to the potential industrial and/or biological hazards that may exist. Many devices have been employed in the prior art to facilitate such capabilities and many attempts have been made that are directed to improving the state of the art.

In the specific example of surgically pertinent medical applications, various medical fluids such as blood, saline, and other corporeal substances must be aspirated or evacuated from an operative site on or in a patient during a surgical procedure so as to keep the surgical field clean and unobstructed. In post-operative wound healing applications, irrigation and bodily waste fluids are often present and must be evacuated to speed recovery and to minimize accumulation of any undesirable substances.

Customarily, a suction canister vacuum pump is used, or a central vacuum pressure source is provided to each of a number of hospital surgical or recovery suites through wall mounted vacuum ports. Such vacuum pressure sources can establish sub-atmospheric pressures or vacuums in the average range of between about 18 and 20 inches of mercury, which can be adjusted or augmented locally to accommodate wound healing drainage applications at vacuums as low as 1.5 inches of mercury, as well as being boosted further to facilitate improved evacuation of smoke and debris using vacuum pressures as high as about 27 inches of mercury or higher.

To prevent fouling and clogging of the pump or the facility-wide vacuum pressure source, waste and excess medical fluids are typically collected locally in each such suite where a procedure is performed using a combination of various types of filtration devices and vacuum bottles and suction canisters. Such bottles and canisters can have wide range of capabilities and capacities and can be adapted to collect and contain such medical fluids and substances during the surgical procedure, and/or during post-operative healing of the surgical site in a manner that establishes safe and sanitary containment and storage retention until post-operative analysis and/or disposal can be accomplished.

Such vacuum bottles and suction canisters can have various configurations and are often arranged as generally cylindrical and sometimes cylindrically conical containers having a lid that closes and seals the container so that a sub-atmospheric pressure or vacuum can be established within the container. The lid may also be adapted to have a suction or vacuum source port, and an aspiration port that is usually connected to a suction tube used to aspirate or evacuate such medical fluids and substances from the operative site and to move them to the interior of the bottle or canister. Other components that can be incorporated into the bottle, canister, and/or lid can include a tandem port for connection to multiple in-line overflow bottles or canisters that can receive such medical fluids and substances after the first such container is filled to capacity. Yet other components and elements can include shut-off valves that can prevent overfilling and spilling and an access aperture that can be adapted to add substances to the contents for treatment and/or solidification, and that can be used to remove the contents of the canister for disposal or testing without the need to remove the lid. One type of such suction canister is described in U.S. Pat. No. 5,470,324 to Cook et al., which is hereby incorporated by reference in its entirety as though fully set forth herein.

In U.S. Pat. No. 6,152,902 to Christian et al., a method and another type of apparatus for collecting surgical fluids is described. The '902 method and apparatus is primarily limited to embodiments that incorporate multiple surgical fluid collection containers that are connected in tandem so that excess and waste fluids evacuated from a surgical site can be safely contained in one or more such containers until the surgical procedure is completed. However, the '902 reference appears to contemplate the possibility that the last container in the tandem configuration can overfill and spill such fluids if the procedure is not completed before that last container is filled.

In U.S. Pat. No. 6,093,230, which is incorporated by reference in its entirety as though fully set forth herein, Johnson et al. describe a filter assembly for use with a suction canister that incorporates two filter elements separated by a hydrophobic foam. The Johnson et al. device is limited to, among other features, capturing airborne contaminants resulting from the aspiration and evacuation of fluids and debris from a surgical site so as to ensure such do not escape the suction canister and escape into the central vacuum pressure source system.

A medical fluid collection canister described by Tribastone et al. in U.S. Pat. No. 5,792,126 is primarily restricted to large volume fluid collection devices and capabilities for a variety of suction, drainage, and specimen collection applications. Tribastone et al. is generally limited to a device that replaces a plurality of smaller canisters with a single larger capacity unit. Another suction canister having a limited use shut-off valve and smoke filter capability is disclosed in U.S. Pat. No. 4,487,606 to Leviton et al., which is incorporated by reference in its entirety as if fully set forth herein. The Leviton et al. device is restricted to, among other elements, a vacuum source port that is in fluid communication with a liquid impervious shut-off valve adapted to interrupt the vacuum port when the canister is filled to capacity. The Leviton et al. device also incorporates a smoke filter that can trap smoke particles generated from laser and electric surgical devices so as to prevent or minimize escape of proteinized smoke vapor particles and fouling of the shut-off valve and the vacuum pressure source system. Another similarly configured suction canister is described in U.S. Pat. No. 4,465,485 to Kashmar et al., which also includes a liquid impervious shut-off valve and filter element that prevents the communication of fluids (liquids and gases) once the canister is filled to capacity with liquid. In U.S. Pat. No. 4,275,732, Gereg also describes a shut-off valve that is limited to, among other features, a liquid impervious membrane that seals the vacuum port once fluid fills the canister and envelopes the membrane.

Other float type mechanical shut-off valves for medical suction canisters on the market use a gasket with a soft rubber gasket material to achieve an effective vacuum shut-off seal. This added gasket component adds complexity and cost to the valve. The present invention (as fully described below) is a superior design than the gasket shut-off valves in the prior art because the present invention achieves the same outcome with no gasket, which eliminates the quality issues surrounding the use of a soft gasket material (e.g. defects, contamination, etc.) and significantly reduces the cost without sacrificing performance.

There have been many attempts to fabricate suction canisters and vacuum bottles that establish or improve the capability to automatically shut-off the vacuum pressure source to prevent overfilling of the particular bottle or canister. Even so, many challenges and problems have persisted and continue to plague the prior art devices. Although many types of such suction canisters have sought to incorporate various means of shutting off the vacuum port, such canisters have demonstrated shortcomings, inefficiencies, and expense.

Notably among the problems in the prior art, it has been found that such shut-off components devices usually produce unpredictable and widely varying results because they are susceptible to unpredictable shut-off valve actuation resulting from poorly fabricated valves and valve components, susceptibility to misalignment of valve components during manufacturing and assembly, susceptibility to fouling of valve components during operation resulting from waste fluids and debris becoming deposited on the valve components, unpredictable valve actuation when subjected to the widely varying vacuum pressures present in the various operational environments and applications, and other short-comings in the physical design of the valve elements that prevent adequate sealing of the valve after actuation. The fouling and misalignment and related problems in particular can create at least two failure modes: first, the valve may not seal properly when actuated such that the vacuum pressure source is not turned off or terminated; second, the valve may stick in a closed position when prematurely actuated due to foam buildup in the canister or bottle from bumping of the canister or turbulent flow within the canister, such that the canister or bottle cannot be filled to capacity without potentially inopportune user intervention.

More specifically, in one exemplary category of prior art problems, the aforementioned membrane shut-off valves can also be susceptible to fouling from smoke and airborne contaminants that may be evacuated or aspirated along with liquids and debris, which contaminants can prematurely and unpredictably clog the fluid impervious membrane or other valve components before the suction canister is filled to capacity. Another category of difficulties and challenges with prior art devices results from the use of various types of elastomeric washers and seals that are employed in attempts to seal the vacuum port when the canister or bottle is filled to capacity. Such washers and seals add complexity and increase manufacturing costs and can further additional failure modes to the valve of the vacuum bottle and suction canister. Additionally, many of the materials available for use as such sealing elements can quickly degrade and become unserviceable and/or fouled during operation, especially when exposed to airborne contaminants such as heated smoke from laser and electric surgical or other tools and instruments.

In further examples of difficulties with prior art devices, the liquid impervious membranes may be afflicted with fabrication defects wherein one or more apertures may develop during the manufacturing or assembly processes, or during actual use, such that fluid communication between the vacuum port and the interior of the suction canister is not interrupted once the canister is filled, which can result in overfilling and spillage. Such undesirable anomalies and performance uncertainty can often require user intervention at inconvenient times and are well-known to those with knowledge in the relevant arts and persists regardless of the desired mode of operation or of the particular type of suction canister or vacuum bottle.

These problems remain in the pertinent field of art and have over time created a long-felt need for advances in the state of the technology to reduce the number and prevalence of such problems so that industrial and medical practitioners can focus more readily on the task at hand without concern for whether the vacuum bottle and suction canister collection tools are functioning as desired. What is immediately needed is a suction canister and vacuum bottle shut-off valve that is less expensive and time consuming to fabricate and that has more predictable results and performance characteristics, and which is readily compatible for use with any of the many types of suction canisters and vacuum bottles presently on the market. The present invention overcomes many of the problems experienced with the prior art devices in a variety of new and novel configurations and with any of a number of possible and equally effective embodiments, configurations, and alternative and preferred arrangements.

### SUMMARY OF INVENTION

In its most general configuration, the present invention advances the state of the art of suction canister shut-off valves with a variety of heretofore unknown configurations that incorporate fewer parts and even eliminate certain parts. Such new configurations and related components are optimized for improved performance under a wide range of operating parameters and environments and are adapted to better resist failure modes and fouling. The markedly innovative enhancements of the present invention include variously adapted embodiments and components that together overcome many of the pitfalls and shortcomings of previously known shut-off valves for use with suction canisters and vacuum bottles in new and novel ways. In one of the many preferable embodiments, and in modifications and alternative configurations thereof, the novel shut-off valves of the present invention establish a significantly higher standard of capabilities and reliability that will in all likelihood minimize if not eliminate prior difficulties with present known suction and vacuum canisters. Such innovative improvements are accomplished with reduced part counts, decreased production costs, and with less operational intervention during use and operation in any of a number of possibly suitable professional, clinical, and home-based medical and healthcare applications as well as various industrial applications.

In one embodiment of a preferred shut-off valve according to the principles of the present invention there is illustrated a gasketless suction canister valve that is compatible for use in any of a variety of applications and suction or vacuum canisters and bottles. The contemplated canisters and bottles can be a fluid collection container that is formed with a periphery defining an opening into the container, and that includes a lid or top that is adapted to be received on the periphery to seal the opening to define an interior of the container.

The top or lid or another portion of the container are also formed to have a suction aperture and vacuum port that are each in fluid communication with the interior of the container. The gasketless valve can be integrally formed as part of or otherwise can be separately formed and incorporated into a top or lid of the suction canister or vacuum bottle.

Among other components and elements, the gasketless valve device has a valve seat that is incorporated with the top or lid and that is adapted with a sealing ramp or inclined valving surface, which presents or projects into the interior of the canister or bottle and is arranged to circumscribe a vacuum source port that is also formed through the lid or top. The gasketless suction canister valve is also further adapted with a valve float that includes a rim or variably graduated sealing float extent that is sized and shaped to be telescopingly received on and to be confrontingly and sealingly seated against the sealing ramp to interrupt and terminate fluid communication through the vacuum port.

The valve float is preferably further formed with at least two differently tapered portions that are configured as part of the rim or variably graduated sealing float extent to seat against the sealing ramp or inclined portion to terminate fluid communication through the vacuum source port to thereby prevent overfilling of the canister during operation and use. Although a large number of possible configurations are contemplated herein, the sealing ramp or inclined portion of the valve seat is preferably formed from polymeric thermoplastic material with a substantially smooth surface finish so that the respective coefficients of sliding and static friction are minimized. In addition to reducing the frictional coefficients, so as to reduce the possibility if not the likelihood of sticking due to fouling of mating surfaces and/or polymeric adhesion between the sealing ramp and the valve float rim, the rim of the valve float is preferably fabricated from a polymeric material that is dissimilar or different from that of the sealing ramp of the valve seat.

In any of a number of variations and alternative configurations, the gasketless valve is a flotation actuated valve device that may optionally be formed with a graduatingly tapered polymeric valve seat that includes a sealing face, both of which are formed about the vacuum source port of the top or lid either integrally or as an attached or fastened component. The flotation actuated valve device further preferably incorporates a valve float that is adapted to have a fluid barrier portion sized and shaped for improved sealing and release capability in a bayonet-type mounting for sealing receipt against the sealing face to, when so mounted and received, stop fluid communication through the vacuum port to disconnect the vacuum pressure source from the interior of the container.

As those skilled and knowledgeable in the relevant and related arts may be able to further contemplate, the preferred and alternative configurations of the embodiments of the present invention are also preferably or optionally adapted to incorporate a large number of possible additional alternative configurations and arrangements that are described in further detail herein below. These variations, modifications, and alterations of the various preferred and alternative embodiments and configurations may be used either alone or in combination with one another as can be better understood by those with skill in the art with reference to the following detailed description of the preferred and optional embodiments and the accompanying figures and drawings.

### BRIEF DESCRIPTION OF THE DRAWINGS

Without limiting the scope of the present invention as claimed below and referring now to the drawings and figures, wherein like reference numerals, and like numerals with primes, if any, across the several drawings, figures, and views refer to identical, corresponding, and/or equivalent elements, components, features, and parts:

FIG. 1 is an elevated isometric generally topside perspective view, in enlarged scale, of a vacuum and suction bottle or canister according to the principles of the present invention;

FIG. 2 is an elevated isometric generally underside perspective view, in modified scale, of the bottle or canister of FIG. 1;

FIG. 3 is an elevated isometric underside view, in enlarged scale and with a portion of the structure removed for purposes of illustration, of a canister lid or top and shut-off valve assembly of the vacuum and suction bottle of FIG. 1;

FIG. 4 is an elevated isometric exploded view, in enlarged scale, of the lid and valve assembly of the vacuum and suction bottle or canister of FIG. 3;

FIG. 5 is a cross-sectional exploded view, rotated and reduced scale, of the lid and valve assembly of FIGS. 3 and 4;

FIG. 6 is a cross-sectional view, in enlarged scale, of a sealing float valve of the valve assembly of FIGS. 3, 4, and 5, which larger scale representation of the sealing float valve is illustrated to depict details, variations, and modifications not susceptible to illustration in the previous smaller scale views;

FIG. 7 is a cross-sectional and partially exploded and partially assembled view, in similar scale, of the lid and valve assembly of FIG. 5;

FIG. 8 is a cross-sectional assembled view, in similar scale, of the lid and valve assembly of FIGS. 3, 4, and 5; and

FIG. 9 is a cross-sectional view, in similar scale, of the lid and valve assembly of FIG. 8 illustrating certain components in operation.

Also, in the various figures and drawings, reference symbols and letters are used to identify significant features, capabilities, dimensions, objects, and relative configurations and arrangements of elements as described in further detail herein below in connection with the several figures and illustrations.

### DESCRIPTION OF THE PREFERRED EMBODIMENTS

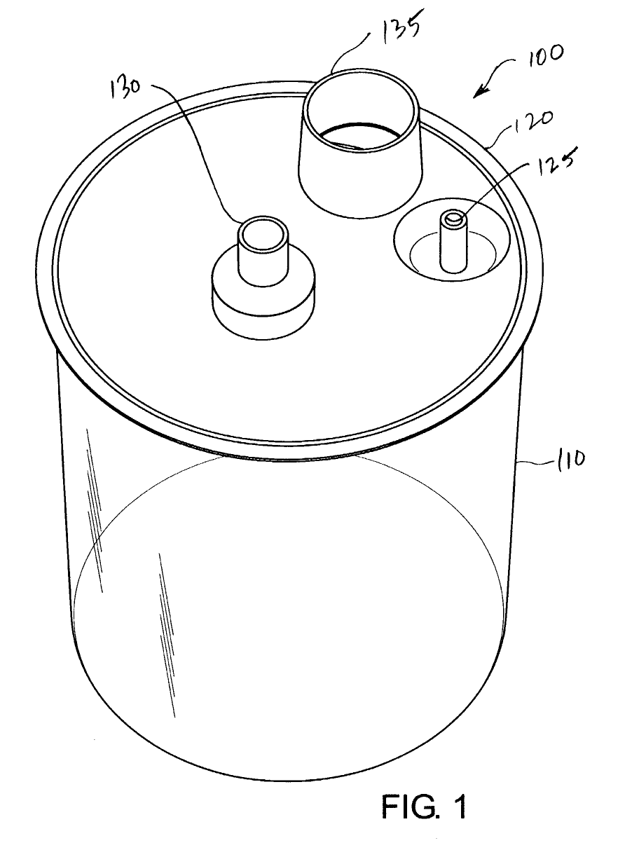

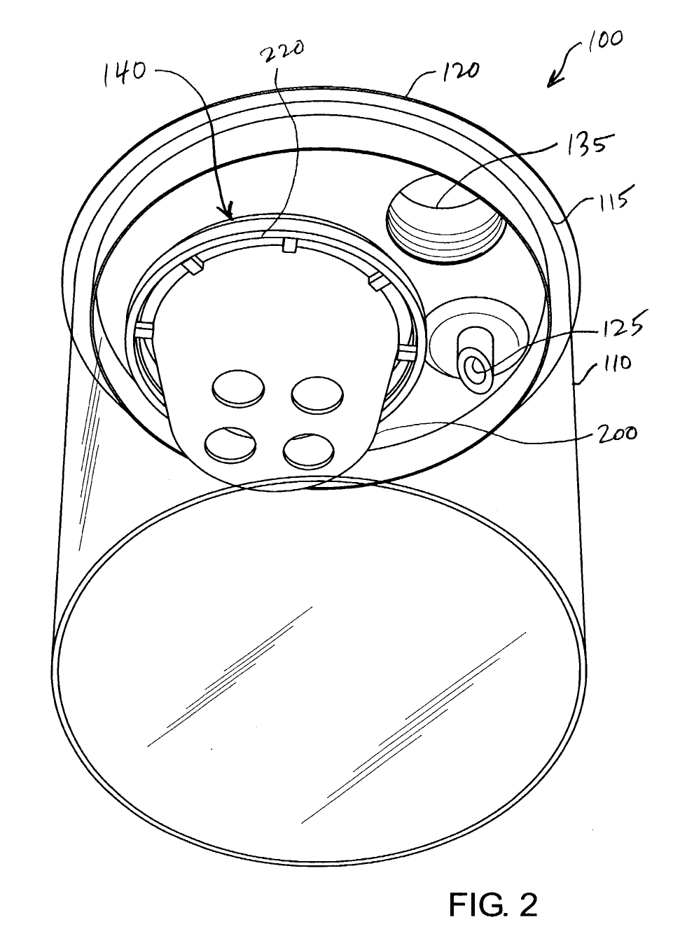

The shut-off valves according to the principles of the present invention are easier and less expensive to manufacture, have few parts, and are more reliable. The shut-off valves can be incorporated into and with suction canisters and vacuum bottles of large and small capacities and that are formed in any number of possible shapes and configurations. The heretofore unavailable capabilities and simplified constructions of the inventive suction canister and vacuum bottle shut-off valves are now described in more detail in the context of the preferred and optional embodiments and modifications and variations thereof. With reference now to FIGS. 1, 2, and 3, the present invention is incorporated into a vacuum bottle or suction canister such as that illustrated and denoted generally in the figures by reference numeral 100. The vacuum bottle and/or suction canister 100 customarily incorporates a fluid collection container 110 that is formed with a periphery 115 (FIG. 2) that forms an opening into the container 110. A lid 120 is also included that is releasably engaged upon the periphery 115 to seal the opening to define an interior of the container 110 so that a vacuum pressure can be established within the container 110.

The container 110, as well as any of the other parts and components of the canister or bottle according to the principles of the present invention as described below, may be formed from any of a number of equally suitable materials that can be selected from the group including metals, glass, ceramics, and polymeric materials that can further include preferably clear and/or translucent materials formed about at least a portion of the container 110 such as, for purposes of example without limitation, thermoplastics including materials selected from any of a variety of commercially available and suitable materials including acetyl resins, delrin, fluorocarbons, polyesters, polyester elastomers, metallocenes, polyamides, nylon, polyvinyl chloride, polybutadienes, silicone resins, ABS (acrylonitrile, butadiene, styrene), polycarbonate, polypropylene, liquid crystal polymers, alloys and combinations and mixtures and composites thereof, and reinforced alloys and combinations and mixtures and composites thereof. Although the container 110 and the lid or top 120 are depicted to have a generally cylindrical upstanding shape, any of a wide range of possible configurations and shapes are contemplated for purposes of the present invention and the cylindrical example of the various illustrations and accompanying descriptions are not intended to in any way limit the preferred shape of the container 110 or the top or lid 120, or of the other components and elements described in connection therewith.

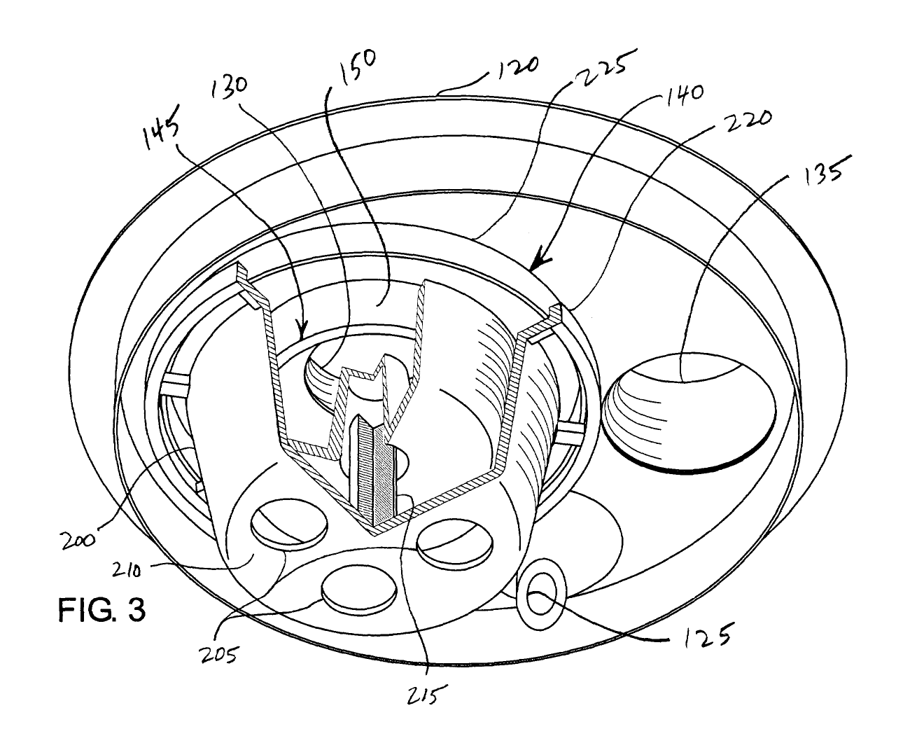

With continued reference to FIGS. 1 and 2, and also specifically to FIG. 3, the top or lid 120, or another portion of the container 110 (not shown), may also be further adapted to incorporate a suction aperture 125 that is in fluid communication with the interior of the container 110 and that can be connected to any of a number of aspiration and evacuation tools and instruments that are known to those skilled in the art for use in removing unwanted fluids including smoke, liquids, and debris from a surgical field or other industrial process and location of interest.

A vacuum port 130 may also be formed in the lid or top 120 to be in fluid communication with the interior of the container 110, and which port 130 is to be connected to a vacuum pressure source (not shown) of the type discussed hereinabove. A tandem connection port 135 may also be formed in the top or lid 120, which can be used to connect the suction canister to additional downstream canisters to capture and retain collected fluids and substances when the container 110 becomes filled to capacity. The tandem connection port 135 may also be used as an infusion port for introducing chemicals such as waster solidifiers and biocides into the collected materials, or as a sampling port so that the collected contents of the container 110 may be tested and/or analyzed. The tandem port 135 may also be further used as a dump port to empty the contents that have been collected in the container 110. Although shown in the various figures as being generally formed in the top or lid 120, the suction, vacuum, and tandem connection ports 125, 130, 135 and related features, elements, and components may also be formed in other various locations and portions of the contemplated vacuum bottle and suction canister 100 as those skilled in the art should be able to comprehend.

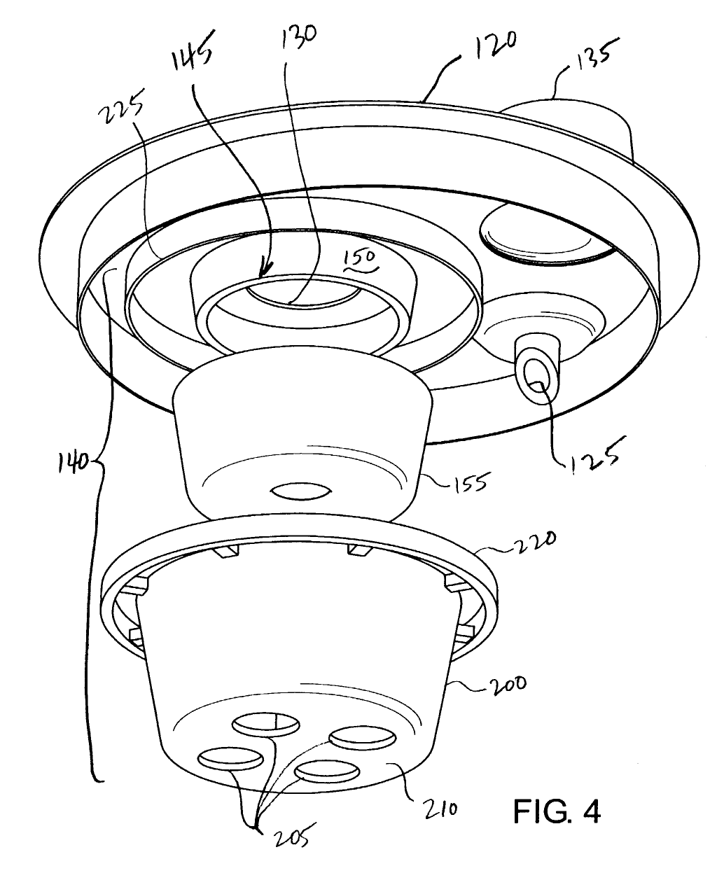

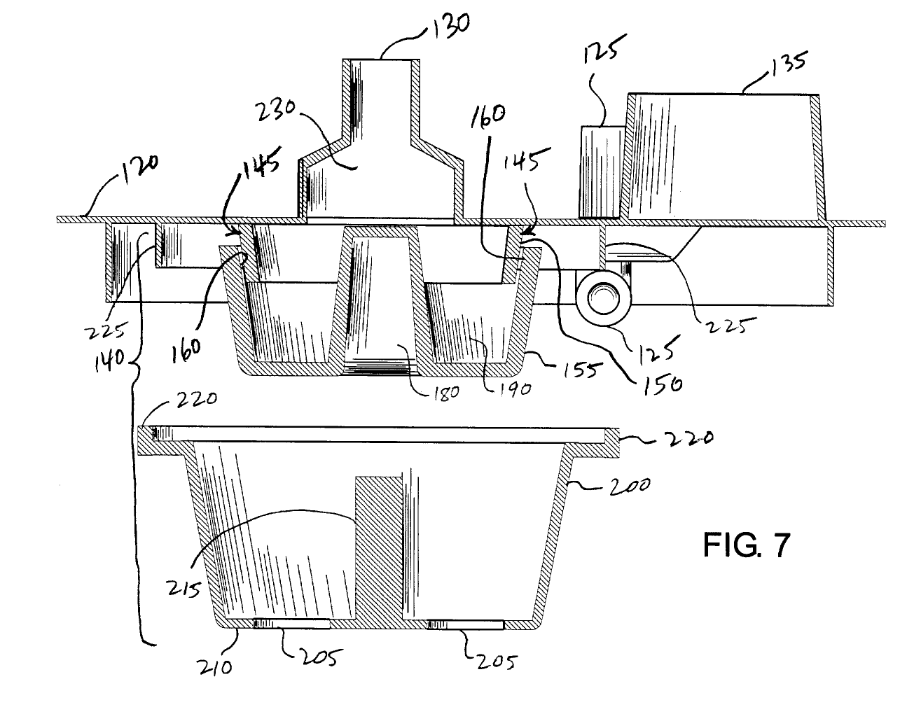

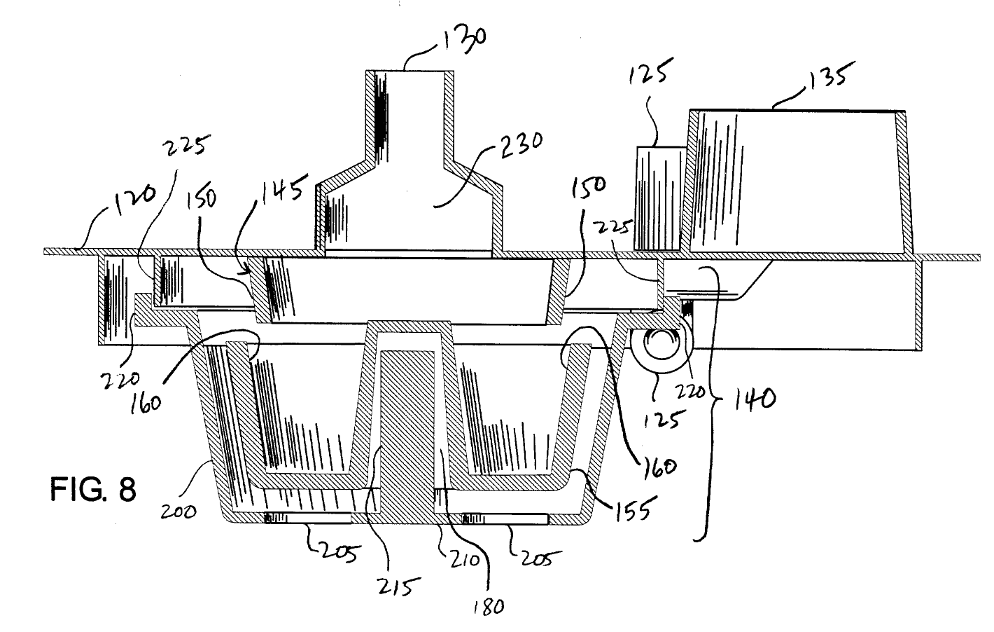

A gasketless valve assembly 140 can be integrally formed as part of, or can otherwise be separately formed and incorporated into and/or permanently or releasably attached to the top or lid 120 of the suction canister or vacuum bottle 100. As shown in the various figures herein, and as illustrated in additional detail in FIGS. 3 through 9, the gasketless valve assembly 140 is depicted as being formed in part integrally with the top or lid 120. The gasketless valve assembly 140 incorporates, among other elements and components, a valve seat 145 that can be optionally or preferably formed as noted or integrally with the top or lid 120. The valve seat 145 preferably surrounds or circumscribes the vacuum port 130.

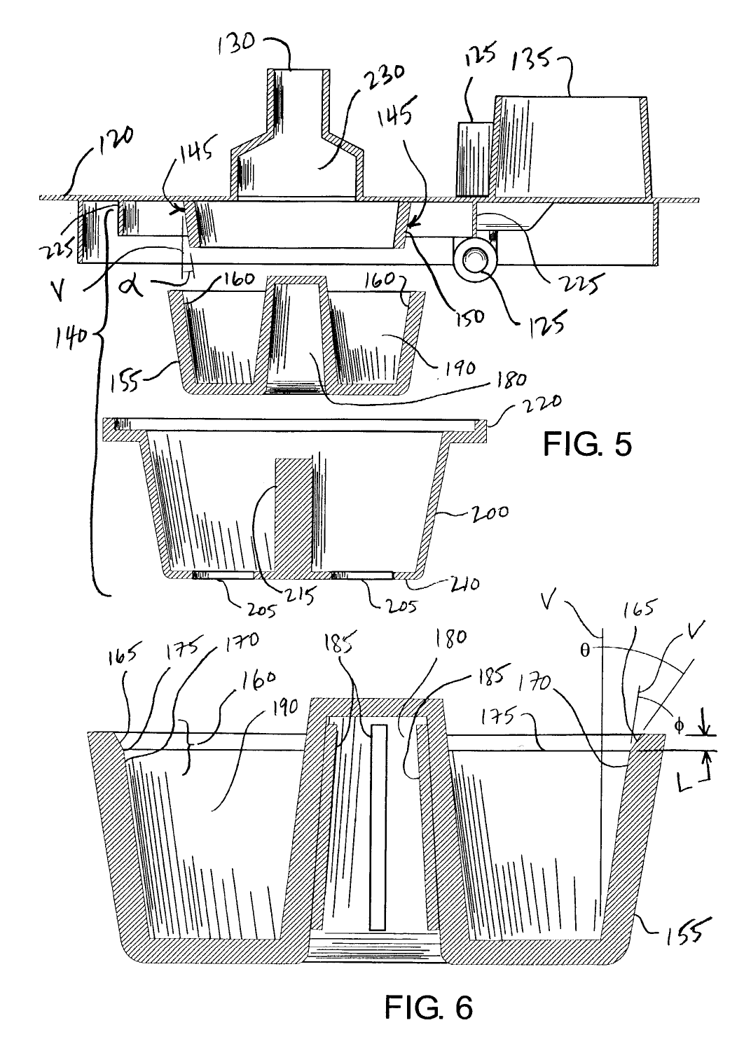

The valve seat 145 is formed with a sealing ramp, sealing face, or inclined valving surface 150 that presents or projects into the interior of the container 110 and which is substantially inclined in cross sectional profile to have one or more inclined angles, such as at least one angle that is denoted generally by reference letter alpha “α” (FIG. 5) that spans between the surface 150 and the approximately longitudinal and/or substantially vertical (relative to the various drawing views) axis “V” (FIG. 5) of the container 110. The valve seat 145 may also be formed as a separate component that can be captured against the lid or top 120 so as to effect the noted capability as can be better understood with continued reference to the additional details of the various embodiments and variations described elsewhere herein.

The gasketless valve assembly 140 also includes a valve or sealing float 155 that is formed to have a multiply tapered rim, fluid barrier portion, or variably graduated sealing float extent 160 that is sized and shaped to releasably, sealing, and confrontingly be seated in a telescoping or bayonet type manner against the sealing ramp, sealing face, or inclined valving surface 150 so as to interrupt and terminate fluid communication through the vacuum port 130 when the container 110 is filled to capacity or to a desired level.

Although not reflected in the generally smaller scale figures, the enlarged scale view of FIG. 6 depicts that the multiply tapered rim, fluid barrier portion, or variably graduated sealing float extent 160 of the valve or sealing float 155 preferably or optionally incorporates multiple tapers or at least two differently tapered portions 165, 170 that are configured as part of the rim or variably graduated sealing float extent 160. The tapered portions 165, 170 are preferably also adapted to have at least two respective substantially inclined unequal angles that span between the surface of the tapered portions 165, 170 and the substantially longitudinal and generally vertical axis V, such as angles phi “φ” and theta “θ” (FIG. 6) that are each also different from angle “α” (FIG. 5) of the sealing ramp, sealing face, or inclined valving surface 150. In this configuration, the multiply tapered rim, fluid barrier portion, or variably graduated sealing float extent 160 can be further defined as or to have an angle or incline transition element formed in the example construction as a generally annular seal 175, which can be formed between the differently tapered portions 165, 170 that can further improve the performance of the sealing function and which can also further augment releasability of the valve or sealing float 155 from the valve seat 145 as discussed in more detail elsewhere herein.

Although the various figures reflect configurations where the multiple tapered portions 165, 170 define and/or are incorporated into the multiply tapered rim, fluid barrier portion, or variably graduated sealing float extent 160 of the valve or sealing float 155, the present invention may also be further modified wherein such multiple tapers are incorporated either alone or combination with tapered portions 165, 170 onto the sealing ramp, sealing face, or inclined valving surface 150 of the valve seat 145. This optional or preferred alternative arrangement modifies the valve seat 145 to establish a graduatingly tapered valve seat (not shown but similar in construction to the multiply tapered portions 165, 170 and that can also optionally have an angle or incline transition element similar to the generally annular seal 175). In this alternative variation, the sealing ramp, sealing face, or inclined valving surface 150 would be further modified to incorporate and/or to define the optionally desirable and respective multiply tapered portions as described.

The valve or sealing float 155 also preferably or optionally incorporates at least one guide keyway 180 having generally inwardly projecting centering rails 185 (FIG. 6), which at least one guide keyway 180 can be adapted in the single keyway configuration shown in the various figures and labeled with reference numeral 180. The valve or sealing float 155 additionally defines a bucket portion 190 that is adapted to displace collected liquids accumulating in the container 110 to buoy the float 155 as the container 110 is filled with the collected liquids. The bucket portion 190 may optionally be replaced and/or augmented with any similarly capable structure or closed-cell material such as a thermosetting urethane or other type of foam that can similarly act to displace the rising collected liquids to buoy the float 155 to actuate the gasketless shut-off valve assembly 140.

A float basket 200 is also included as part of the gasketless valve assembly 140 to capture the valve or sealing float 155 as shown in the various figures proximate to the lid or top 120. The float basket 200 incorporates one or more apertures 205 formed in the floor 210 or other wall to communicate collected and rising fluids between the interior of the container 110 and the underside of the buoyant valve or sealing float 155. The float basket 200 also may include at least one generally upwardly projecting guide key or post 215 that is adapted to cooperate with the guide keyway 180 and the centering rails 185 of the valve or sealing float 155 as the float 155 is actuated by the buoyant force resulting from the displaced collected fluids rising in the container 110.

For configurations of the gasketless valve assembly 140 that are adapted for interchangeability of the components such as the valve or sealing float 155, the float basket 200 can be further adapted, for purposes of further illustrations and examples without limitation, to be releasably snapped into place against the top or lid 120 with a frictional fitting snap element 220 that can be formed about a superior portion of the float basket 200. The snap element 220 can be generally ring shaped to cooperate with a correspondingly adapted basket seat 225 that can be formed integrally in the top or lid 120. In this optional or preferable modification, which can be incorporated into any of the preceding embodiments or variations of the present invention, the valve or sealing float 155 can be interchanged with variously optimized alternative floats to establish predetermined performance characteristics and to accommodate specialized automated shut-off compatibility with a variety of fluids and materials to be collected during operation and use of the proposed vacuum bottle and suction canisters 100.

In operation, it has been established that the various configurations of the present invention offer significant advantages over prior devices wherein the valve and sealing float 155 resists misalignment or fouling induced non-sealing as well as sticking and adhesion. The improved design consistently operates to positively seal the vacuum port 130 to interrupt and terminate fluid communication therethrough as the level of collected fluids rises to capacity within the container 110. Moreover, the valve or sealing float 155 releases and unseals to reinitiate fluid communication through the vacuum port 130 as the fluid level in the container 110 subsides such as when the container 110 is emptied or as possible foam buildup is dissipated. Although not shown in the figures, the valve and sealing float may be further adapted to be precisely weighted to overcome such foaming induced premature actuation circumstances.

These advanced and improved automatic shut-off and release capabilities are accomplished without the use of gaskets and gasket type materials by way of the described constructions, which embodiments and variations each incorporate one or more multiply tapered portions one or more of the various surfaces as described and depicted. While a wide range of possible taper angles have been found to be satisfactory for purposes of the present invention, it has been demonstrated that many optimal and specific configurations are well-suited for purposes of vacuum bottles and suction canisters, such as bottle or canister 100, that are to be used for purposes of collecting fluids in various industrial and medical applications. More specifically, satisfactory operational characteristics including adequate sealing and shut-off, and consistent non-sticking and release and unsealing are achieved when the relative tapers or inclinations between the inclined portion of the sealing ramp, sealing face, or inclined valving surface 150, have angles denoted generally by reference symbol α (FIG. 5), that are dissimilar to the angles denoted by reference symbols φ and θ (FIG. 6) of the multiple tapered portions 165, 170. Even more specifically, satisfactory results are obtained when θ is selected to be approximately less than α, and α is selected to be approximately less than φ in all material tolerance conditions, namely in the best and worst, or maximum and minimum, material conditions.

For purposes of additional illustrations, but not for purposes of limitation, the various example configurations and alternatives depicted in the figures and described in the accompanying text herein have been found to operate satisfactorily when the angle θ of the sidewall tapered portion 170 of valve or sealing float 155 is selected to be approximately between 0 (zero) and 10 degrees of arc, and more preferably between about 3 and 7 degrees, and even more preferably about 5 degrees from a generally longitudinal or substantially vertical direction denoted in the various figures by reference letter V (FIGS. 5 and 6).

The compatibly configured tapered portion 165 can have an angle φ that is preferably between about 13.5 and 20 degrees or greater, and more preferably in the range of between about 13.5 and 16 degrees, and even more preferably approximately between 14 and 15 degrees. The mating valve seat 145 can be formed with a sealing ramp, sealing face, or inclined valving surface 150 having an incline angle α that is preferably in the range of approximately 10 and 13.5 degrees, and more preferably in the range of between about 11 and 13.5 degrees, and even more preferably approximately 13 degrees.

Further, the tapered portion 165 that is inclined at the preferred angle φ has been found to perform well with a substantially vertical or longitudinal dimension L (FIG. 6) in the range of approximately 0 (zero) and 0.250 inches, and more preferably between about 0.005 inches and 0.100 inches, and even more preferably between about 0.020 inches and 0.080 inches, and most preferably about 0.050 inches. The transition between the angle of inclination of the tapered portion 165 and the tapered portion 170 results in the angle or inclination transition portion that is formed in the exemplary and illustrative variations as the generally annular seal 175. The angle or inclination transition portion or generally annular seal 175 can be further modified to have a cross-sectional profile that ranges from a substantially sharp point to a generous and gradual radius. The specific profile of the seal 175 can be adjusted according to the materials used to fabricate the interfacing components and according to the properties and characteristics of the intended operating environment that can include the fluids (gases and liquids) and materials to be collected, and the range of operating vacuum pressures. Similarly, the various angles and dimensions are described for purposes of illustrating one possible embodiment and arrangement of elements and features that demonstrated the new and innovative technology that is incorporated in the proposed gasketless valve assembly 140. However, many such angles and dimensions can be suitable for implementing the technology of the present invention and will depend, among other parameters, upon 1) the materials used to fabricate the sealing and mating surfaces, the application environment wherein the valve is to be used (i.e. vacuum pressure source and substances to be collected), the dimensions of the gasketless valve assembly to be incorporated, and the shape and capacity and throughput of the vacuum bottle or canister.

Additional improvements in the various noted performance characteristics have been also demonstrated in configurations of the present invention that incorporate, among other features and properties, the valve seat 145 being fabricated from a first material and the multiply tapered rim, fluid barrier portion, or variably graduated sealing float extent 160 of the valve or sealing float 155 being constructed of a non-sticking similar and/or non-sticking dissimilar material. Contemplated dissimilar material combinations can include, for purposes of example without limitation, glass and polymeric materials such as thermoplastics, metal and polymeric materials such as thermoplastics, and various dissimilar polymeric materials including for example, thermoplastics that can establish a plastic to plastic interface of compatible but dissimilar thermoplastics or even similar but non-sticking thermoplastics.

More particularly by way of exemplary illustration, satisfactory sealing and releasing of the valve or sealing float from the valve seat 145 has been evidenced under a range of operating sub-atmospheric vacuum operating pressures ranging between about 1.5 and 28 inches of mercury for a wide range of valve compatible similar and dissimilar materials. In one example arrangement, for purposes of illustration only but not for purposes of limitation, the material of the sealing ramp, sealing face, or inclined valving surface 150 of the valve seat 145 was selected to be a polymeric thermoplastic such as a polystyrene or an ABS, which can also be used to form the optionally integral lid or top 120. In the same example configuration, the multiple tapered portions 165, 170 of the multiply tapered rim, fluid barrier portion, or variably graduated sealing float extent 160 of the valve or sealing float 155 can be formed from a polymeric thermoplastic material such polypropylene, among other similarly capable materials that are non-sticking and that freely release from the mating surface(s) of the valve seat 145.

Even further enhanced performance characteristics have been evidenced wherein the surface finishes of the respective mating surfaces between the valve seat 145 and the valve or sealing float 155 are adjusted to have a respective surface roughness that is generally considered by those having skill in the art to be characterized as smooth. More particularly, the surface roughness of the sealing ramp, sealing face, or inclined valving surface 150 and the multiple tapered portions 165, 170 can be adjusted by defining a suitable injection mold surface finish that will be imparted to the polymeric molded material. The most effective substantially smooth surface finishes can be in the range of about substantially 0 (zero) and 500 microinches, and more preferably between approximately 25 and 250 microinches, and even more preferably between about 80 and 150 microinches, and most preferably about 125 microinches. More particularly, the surface roughness of the sealing ramp, sealing face, or inclined valving surface 150 and the multiple tapered portions 165, 170 can be in the range of surface finishes specified as a function of the injection mold finish, which finishes have been standardized by the Society of the Plastics Industry, Inc., (“SPI”) which is headquartered at 1801 K Street, N.W., Suite 600K, Washington, D.C., 20006, USA, and that has an internet web-site at www.socplas.org, and which can have a mold surface finish in the range of smooth finishes specified by the SPI as SPI-A2 to about SPI-D1.

In yet additional alternative and modifications of any of the previously described embodiments and configurations, the total surface area of the mating surfaces of the gasketless valve assembly 140 can be adjusted by changing the relative dimensions of the various structures to vary the contact forces across the interface of the mating surfaces. Such adjustments can further optimize the performance and operational capabilities of the gasketless valve assembly 140 so as to ensure satisfactory operation under unexpectedly abnormal and/or nominally relatively low and high operating vacuum pressures as well as in the presence of a wide range of collected fluids and substances and materials.

Generally, satisfactory sealing forces have been achieved using the relative dimensions possible for the contemplated embodiments and variations thereof of the gasketless valve assembly 140 described herein wherein the surface area of the mating interface between the valve seat 145 and the valve or sealing float 155 has been substantially maximized. More specifically, a total surface area of the mating sealing surfaces of the gasketless valve assembly 140 that has performed well can preferably be between about 1.0 and 2.0 square inches, and more preferably in the range of about 1.5 and 1.9 square inches, and even more preferably approximately 1.75 square inches. These example dimensional ranges have been found to be suitable for purposes of use with vacuum bottles and suction canisters used in nominal medical environment vacuum pressures and that are presently available from a variety of suppliers including, for purposes of example without limitation, Allegiance Healthcare Corp., of McGaw Park, Ill., USA, which has an internet web-site at www.allegiancehealth.com, offers a range suitably adaptable suction canisters, canister kits, and related components that are offered under the brand names Medi-Vac®, Flex Advantage®, Vac-Rite®, Guardian™, and CRD™ brand and which are all compatible for use with the embodiments of the present invention.

In yet other alternative arrangements, various airborne particle filtration elements can be incorporated that can further augment the existing capabilities wherein one or more filtration elements (not shown) can be received within the gasketless valve assembly 140 in a generally superior orientation thereto and within the lumen of the vacuum port 130, which may be further adapted, among other possible modification, to have filter recess 230.

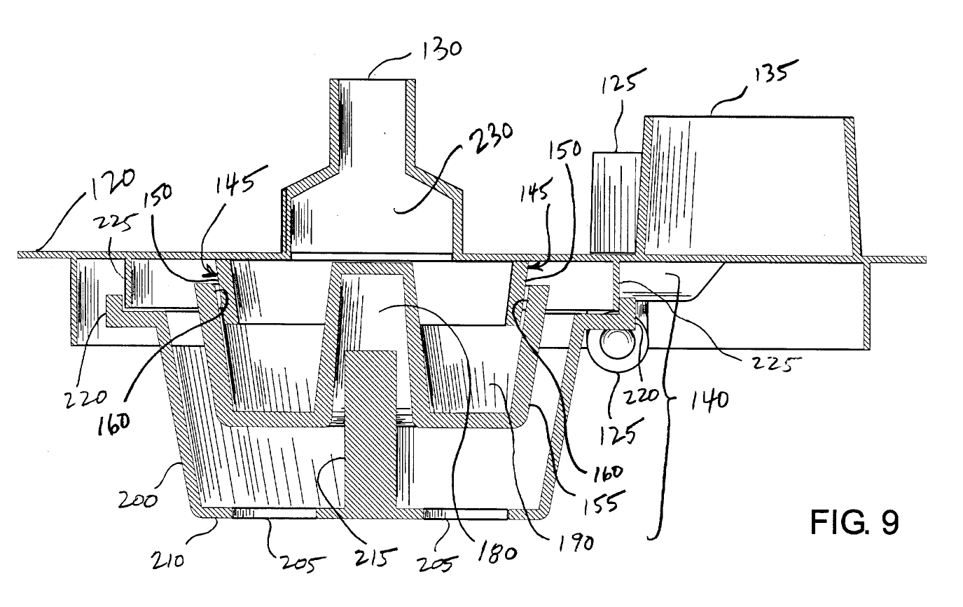

In operation, with reference now specifically to FIGS. 8 and 9, those having skill in the art may be able to comprehend that the valve or sealing float 155 assumes an at rest orientation in the float basket 200 as depicted in FIG. 8 when the container 110 has yet to be filled to capacity. As the container 110 is filled with collected fluids and materials that have been communicated into the interior of the container 110 under the vacuum pressure through suction aperture 125, the fluid level rises in the container 110 as well as in the float basket 200 so that the valve or sealing float 155 is buoyed and rises to confront and to matingly and sealingly seat against the valve seat 145, which thereby interrupts and terminates fluid communication of gases through the vacuum port 130 such that the vacuum pressure source no longer can communicate with the interior of the container 110. As the liquid level in the container subsides, the valve or sealing float then releases from the valve seat 145 and fluid communication through the vacuum port 130 is reestablished and a sub-atmospheric vacuum pressure is again developed in the container 110.

Numerous alterations, modifications, and variations of the preferred embodiments disclosed herein can be understood by those having skills in the various related technical fields of endeavor. All such embodiments contemplated to be within the spirit and scope of the present invention, which is intended to be limited only by the following claims. For example, although specific embodiments have been described in detail, those with skill in the art can understand that the preceding embodiments and variations can be modified to incorporate various types of substitute and/or additional materials, relative arrangements and alternative configurations of elements, and myriad possible dimensional configurations for compatibility with the wide variety of possible vacuum and suction bottles and canisters that are presently known in the prior art and in widespread use in various fields, including medical and industrial fields. Accordingly, even though only few embodiments, alternatives, variations, and modifications of the present invention are described herein, it is to be understood that the practice of such additional and alternatively preferable modifications and variations and the equivalents thereof, are intended to be within the spirit and scope of the invention as defined in the following claims.

What is claimed is:

1. A gasketless suction canister valve, comprising:
a canister lid incorporating a valve seat formed to have a sealing ramp surrounding a vacuum source port; and
a valve float formed to include a multiple tapered rim configured to be telescopingly received on the sealing ramp to interrupt fluid communication through the vacuum source port.

2. The gasketless suction canister valve according to claim 1, wherein the valve seat is formed to have a substantially smooth surface finish from a polymeric material and wherein the valve float is fabricated from a different polymeric material.

3. The gasketless suction canister valve according to claim 1, wherein the valve seat is formed from a substantially low surface roughness polystyrene and the valve float rim is formed from a substantially low surface roughness polypropylene.

4. The gasketless suction canister valve according to claim 1, wherein the valve seat is configured to have at least one first taper and the valve float rim is formed with a second taper greater than the at least one first taper.

5. The gasketless suction canister valve according to claim 4, further comprising a third taper less than the at least one first and the second tapers.

6. The gasketless suction canister valve according to claim 1, wherein the valve seat is adapted to have multiple taper angles different from the angles of the tapers of the multiply tapered float valve rim.

7. The gasketless suction canister valve according to claim 1, wherein the valve seat and valve float are configured to be incorporated into a fluid collection suction canister and to be actuated with a vacuum of approximately between 1 and 28 inches of mercury when a collected fluid level rises sufficiently to buoy the valve float against the valve seat.

8. The gasketless suction canister valve according to claim 1, wherein the valve seat and valve float rim generally form a sealed scarf-type joint that prevents fluid communication through the vacuum source port.

9. A suction canister, comprising:
a fluid collection container formed with a periphery that defines an opening into the container;
a lid adapted to be received on the periphery to seal the opening;
a suction aperture and vacuum port formed in the lid and each in fluid communication with an interior of the container; and
a gasketless valve assembly in fluid communication with the vacuum port and adapted with an inclined valving surface surrounding the vacuum port and configured to confrontingly seat a variably graduated sealing float extent to stop fluid communication through the vacuum port.

10. The suction canister according to claim 9, wherein the inclined valving surface is formed from a polymeric material and wherein the sealing float extent is fabricated from a different polymeric material.

11. The suction canister according to claim 9, wherein the valving surface is formed from a substantially low surface roughness polystyrene and the sealing float extent is formed from a substantially low surface roughness polypropylene.

12. The suction canister according to claim 9, wherein the valving surface is configured to have at least one first taper and the valve float rim is formed with a second taper greater than the first taper.

13. The suction canister according to claim 12, wherein the valve float rim further comprises a third taper less than the first and second tapers.

14. The suction canister according to claim 9, wherein the valving surface is adapted to have multiple taper angles different from the angles of the varied graduations of the variably graduated sealing float extent.

15. The suction canister according to claim 9, wherein the valving surface and the variably graduated sealing float extent are configured to be actuated with a vacuum of approximately between 1 and 28 inches of mercury when a collected fluid level rises to buoy the sealing float extent against the valving surface.

16. The suction canister according to claim 9, wherein the valving surface and the sealing float extent generally form a sealed scarf-type joint that prevents fluid communication through the vacuum source port.

17. A flotation actuated valve device, comprising:
a graduatingly tapered polymeric valve seat formed about a vacuum source port with a sealing face; and
a valve float configured with a fluid barrier portion for bayonet receipt against the sealing face to terminate fluid communication through the vacuum port.

18. The flotation actuated valve device according to claim 17, wherein the sealing face of the valve seat is formed with a substantially smooth surface finish and wherein the fluid barrier portion of the valve float is fabricated from a dissimilar polymeric material.

19. The flotation actuated valve device according to claim 17, wherein the sealing face of the valve seat is formed from a substantially low surface roughness polystyrene and the valve float barrier portion is formed from a substantially low surface roughness polypropylene.

20. The flotation actuated valve device according to claim 17, wherein the valve seat sealing face is configured to have at least one first taper and the valve float barrier surface is formed with a second taper greater than the at least one first taper.

21. The flotation actuated valve device according to claim 20, wherein the valve float barrier surface is also formed with a third taper less than the at least one first and the second tapers.

22. The flotation actuated valve device according to claim 17, wherein the valve seat sealing face is adapted to have multiple taper angles different from the angles of the tapers of the valve float barrier surface.

23. The flotation actuated valve device according to claim 17, wherein the valve seat and valve float are configured to be incorporated into a fluid collection suction canister and to be actuated with a vacuum of approximately between 1 and 28 inches of mercury when a collected fluid level rises sufficiently to buoy the valve float against the valve seat.

24. A gasketless suction canister valve, comprising:
a canister lid incorporating a valve seat formed to have a sealing ramp surrounding a vacuum source port, wherein the valve seat is configured to have at least one first taper and the valve float rim is formed with a second taper that is different than the first taper; and
a valve configured to be telescopingly received on the sealing ramp to interrupt fluid communication through the vacuum source port.

25. The gasketless suction canister valve according to claim 24, wherein the valve seat is formed to have a substantially smooth surface finish from a polymeric material and wherein the valve float is fabricated from a different polymeric material.

26. The gasketless suction canister valve according to claim 24, wherein the valve seat is formed from a substantially low surface roughness polystyrene and the valve float rim is formed from a substantially low surface roughness polypropylene.

27. The gasketless suction canister valve according to claim 24, further comprising a third taper less than the at least one first and the second tapers.

28. The gasketless suction canister valve according to claim 24, wherein the valve seat is formed to include a multiple tapered rim.

29. The gasketless suction canister valve according to claim 24, wherein the valve seat and valve float are configured to be incorporated into a fluid collection suction canister and to be actuated with a vacuum of approximately between 1 and 28 inches of mercury when a collected fluid level rises sufficiently to buoy the valve float against the valve seat.
